(19) United States
(12) Patent Application Publication
(10) Pub. No.: US 2007/0057086 A1
(43) Pub. Date: Mar. 15, 2007

(54) Replenishable dispensing device for fragrance

(76) Inventor: Wijnanda Hendrika Van Kippersluis, Locust, NJ (US)

Correspondence Address: INTERNATIONAL FLAVORS & FRAGRANCES INC., 521 WEST 57TH ST, NEW YORK, NY, 10019, US

(21) Appl. No.: 11/223,731

(22) Filed: Sep. 9, 2005

(51) Int. Cl. A61L 9/04; A24F 25/00

(52) U.S. Cl. 239/43; 239/34; 239/37

(57) ABSTRACT

The present invention provides a fragrance dispensing device, which is gravity-induced and capable of replenishing the fragrance at will by the use of a gravity-induced supply. More particularly, the present invention relates to a cartridge type device which contains a fragrance. The air freshening cartridge can be an independent unit or can be incorporated into the design of consumer products so that as a consumer product is tilted or inverted during normal use the dispensing device is refreshed with flowing liquid or gel fragrance.

### BACKGROUND OF THE INVENTION

Traditional air freshening dispensers employ a wick to convey a liquid or a volatile material from a container to an emanator and are designed to slowly diffuse a liquid through the wick. The liquid may be a volatile scent-producing liquid, as in the case of air fresheners. In wick type air fresheners, the scent-producing liquid is conveyed from a container, by means of capillary action through a wick, to an emanator, which diffuses the scent-producing liquid into the atmosphere in the form of a vapor, by the process of evaporation. Wick type liquid dispensers, which diffuse liquid material into the atmosphere, at a slow, controlled rate, are not limited to use as air freshening devices.

While wick type liquid dispensing devices are applicable to many different uses with minor modifications, wick type liquid dispensers are especially suited, and commonly used for stand alone air freshening devices. When used in this manner the air fresheners require a space in which to rest on a table or shelf. Further, in order to be effective, the wick type dispensing device must provide the desired fragrance at a slow uniform rate, over an extended period of time.

U.S. Pat. No. 6,435,423 discloses a device for the diffusion of a volatile active ingredient with a hydrophobic medium, a volatile active ingredient and a superabsorbent substance. The superabsorbent substance is capable of forming a gel with the hydrophobic medium and is susceptible of enclosing the volatile active ingredient within the gel to permit diffusion of the volatile ingredient from the gel upon exposure to air. The device enables a fresh supply of fragrance composition to be fed to the emanator surface each day preventing gradual diminution in the rate of emanation of the fragrance composition with time.

U.S. Pat. Nos. 4,549,693 and 4,306,679 disclose dispensers comprised of a two part housing, whereby the effective aperture openings in the housing are controlled by twisting or sliding motion, bringing the apertures in each part, in and out of register.

U.S. Pat. Nos. 6,569,387 and 6,610,254 disclose a dual function dispenser for releasing a long-term passive or continuous release of a dispersible composition, or an active release such as a burst or spray of the chosen composition.

U.S. Pat. No. 6,699,432 discloses a fragrance emanating device for freshening atmospheric air comprising a closed container for accommodating a liquid fragrance composition in its interior and emanator having a surface open to the ambient air for evaporation of the liquid fragrance composition and a conduit linking the emanator surface with the liquid fragrance composition.

U.S. Pat. No. 6,703,011 discloses a fragrance control release system which is an emulsifier-free single phase, non-porous continuous permeable polymeric film having a substantially uniform thickness of from about 1 up to about 150 microns, having entrapped and dissolved therein molecules of at least one fragrance substance capable of evolving from the film into the environment.

A basic disadvantage associated with the above described dispensers is the requirement for a user to continually adjust the housing aperture to maintain a desired scent level. A further disadvantage is the limited supply of scent-producing liquid which is subject to a shortened period of effectiveness depending upon the environment and liquid absorbing capabilities of the absorbent. Wick or film types of air freshener units have traditionally suffered from a lack of adequate control, to deliver a scent-producing liquid at a slow, constant rate over an extended period of time.

There remains a need for an improved dispensing device, capable of slow, controlled dispensing and diffusion of fragrances. The primary advantage of the present invention is the slow, controlled dispensing of an active liquid material to a location by a use of a replenishable supply cup and an emanator. The active liquid or fragrance is continually refreshed to the emanator as the commercial good is used.

### SUMMARY OF THE INVENTION

It is an object of the present invention to provide a fragrance dispensing device comprising a reservoir or a supply cup which holds an active liquid, and an emanator. The supply cup maintains the active liquid in isolation from the ambient atmosphere and replenishes the active liquid of the emanator. The emanator receives active liquid from the supply cup via a tubular element, the emanator diffuses the active liquid into the ambient atmosphere by the process of evaporation. The active liquid is desirably a fragrance; however, the active liquid can be any desired air dispersible medium, for example, a nutrient or insect repellant. The dispensing device can further comprise an adhesive backing so that the dispenser can be adhered to a surface.

It is another object of the present invention to provide a dispensing device comprising a supply cup which holds an active liquid, an emanator, and a holding vessel used to store consumer products. The supply cup maintains the active liquid supply in isolation from the ambient atmosphere and replenishes the active liquid of the emanator. The emanator receives active liquid from the reservoir via a tubular element and the emanator then diffuses said active liquid into the ambient atmosphere by the process of evaporation. The holding vessel used to store consumer products can be formed into any number of desirable configurations.

Another object of the present invention is a method for providing an active liquid to the environment comprising tilting a dispensing device comprising a supply cup which holds an active liquid and an emanator. Upon tilting the dispensing device, the emanator receives active liquid from the supply cup via a tubular element and the emanator then diffuses said active liquid into the ambient atmosphere by the process of evaporation.

### BRIEF DESCRIPTION OF THE DRAWINGS

FIG. 1 depicts an exploded view of the present dispensing device.

FIG. 2A depicts a head on view of a second embodiment of the present invention.

FIG. 2B shows an exploded view of a second embodiment of the present dispensing device.

FIG. 3A depicts a head on view of a third embodiment of the present invention.

FIG. 3B depicts a head on view of a third embodiment of the present invention.

FIG. 3C depicts a head on view of a third embodiment in the engaged position of the present invention.

FIG. 3D depicts a head on view of a third embodiment of the present invention.

### DETAILED DESCRIPTION OF THE INVENTION

The main advantage of the present invention is that it provides long-lasting fragrance performance. There is an equivalent amount of fragrance supplied as desired or alternatively every time a consumer goods bottle is tilted or otherwise moved to fill the dosing chamber.

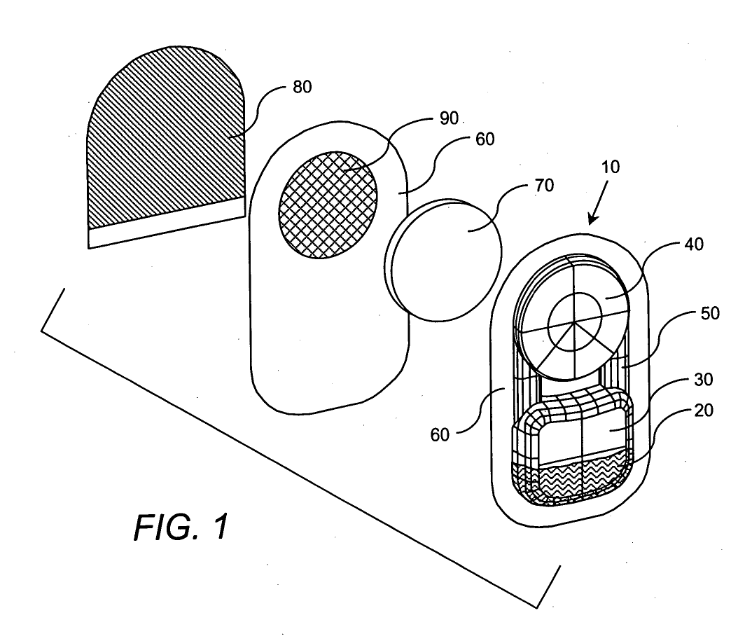

FIG. 1 shows an exploded view of the dispensing device comprising a dispensing device of the invention 10 which holds an active liquid 20, wherein said dispensing device 10 maintains the active liquid 20 in isolation from the ambient atmosphere; a supply cup 30 located within the dispensing device 10; and an emanator 40 which receives active liquid 20 from the supply cup 30 via a tubular element 50, said emanator 40 diffuses said active liquid 20 into the ambient atmosphere by the process of evaporation.

The dispensing device of the present invention 10 comprises a supply cup 30 and an emanator 40 for dispersing fragrance from an active liquid. The dispensing device 10 holds an active liquid, such as a fragrance. The dispensing device 10 holds or maintains the active liquid 20 supply in isolation from the ambient atmosphere so that it does not leak or evaporate. A supply cup 30 is located within the dispensing device 10 so that the emanator 40 can be replenished with active liquid 20 from the supply cup 30 as needed or desired. An emanator 40 receives active liquid 20 from the supply cup 30 via a tubular element 50. The emanator 40 diffuses the active liquid 20 into the ambient atmosphere by the process of evaporation.

The dispensing device 10 comprises a generally planar bag formed by the peripheral union of two wall members 60, said wall members being comprised of an impermeable material and being sealed together in a continuous manner along their edgewise periphery providing a liquid-tight peripheral seal, wherein a passageway extends from said supply cup 30 through said peripheral seal to accommodate the protrusion of said tubular element 50 between said wall members, wherein a liquid-tight seal is additionally maintained between said periphery seal and said emanator 40. The dispensing device 10 can comprise an impermeable flexible material such as foils, metals, polymeric films, polyethylenes, polypropylenes, polyvinyl chlorides, polyacrylates, polyamides, polyesters, polycarbonates, polyvinylidene chlorides, ethylene vinyl acetates, polystyrenes, modified polystyrenes, polyfluorocarbons, metallized polymeric films, composite laminates or coextrusions of such aforementioned films. The dispensing device 10 can further comprise an adhesive backing and a release layer wherein the release layer can be removed to expose said adhesive backing so that the dispenser can be adhered via the wall of the dispensing device 10 to a surface such as a bottle, box, sporting good, consumer good, package, or other desirable surface.

The emanator 40 is replenished with active liquid 20 when the dispensing device 10 is rotated or tilted so that the active liquid 20 flows from the supply cup 30. After the emanator 40 is filled the excess active liquid 20 is recaptured and held in the supply cup 30 ready to replenish the emanator 40 as needed.

The emanator 40 is supplied with active liquid 20 from the supply cup 30 via a tubular element 50 protruding through said dispensing device 10 container wall. The emanator 40 makes physical contact with said active liquid 20 during functional operation, and said emanator 40 comprises an absorbent pad 70 such as paper, cloth, filter paper, or other desirable material. The tubular element 50 is open at each end and connects the emanator 40 with the supply cup 30. The tubular element 50 can be an actual hollow component or can comprise simply a pierced opening or passage which allows free flow of the active liquid 20. The tubular element 50 allows the active liquid 20 to flow through the tubular element 50 and make physical contact with the emanator 40. During functional operation said supply cup 30 on the other end makes physical contact with said active liquid 20 provided to the supply cup 30.

The emanator 40 comprises an absorbent material 70 capable of absorbing said active liquid 20 and releasing said active liquid 20 into the ambient atmosphere by the process of evaporation. The absorbent material can comprise high porosity fibers or high porosity cellulose, woven cotton fibers, non-woven cotton fibers, woven synthetic fibers, non-woven synthetic fibers, polymeric foams, porous ceramics, paper, cloth filters, sponges, or any other suitable materials. Optionally, the emanator 40 can further comprise a peelable seal 80 which can be removed to expose the emanator 40 to the atmosphere. The vent 90 is depicted opposite to the emanator 40 wherein the absorbent material 70 is placed between the emanator 40 and the vent 90. The vent 90 may have the same shape as the absorbent material 70 and the emanator 40 as depicted in FIG. 1.

The active liquid 20 is desirably a scent-producing oil, scent-producing liquid, scent-producing gel, scent-producing thickened fragrance or an insect repellent being maintained isolated from said emanator 40 prior to functional operation by means of a containment member such as a stopper, whereby upon removal of said containment member said active liquid 20 is free to contact said emanator 40 thus, commencing the functional operation of said active liquid 20 dispensing device.

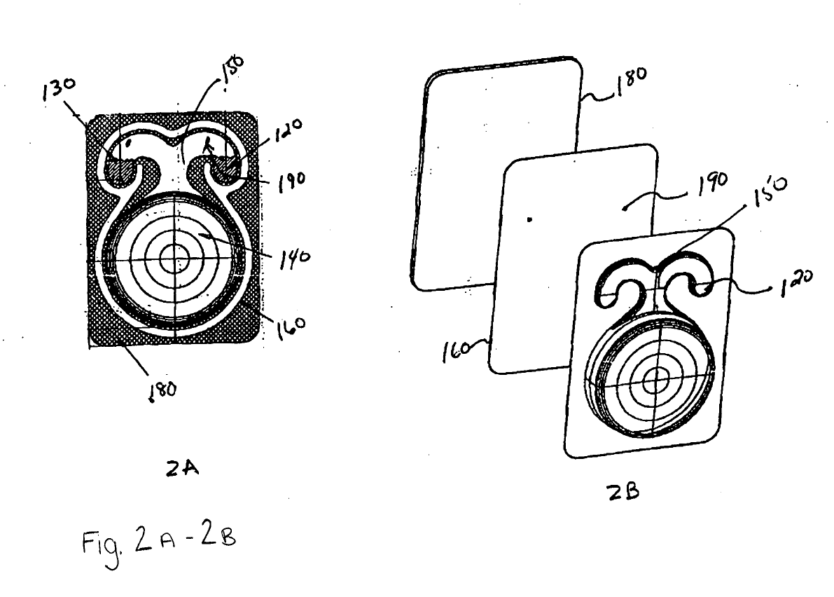

Referring to FIGS. 2A and 2B, a second embodiment of the present invention is presented. In this embodiment the active liquid is provided in one or more supply cups 130 which are formed in a weir shape device wherein the liquid level is below the fluid connecting tubes 150 which provides liquid communication between the supply cups and the absorbent pad and emanator below. The active liquid is dispensed by tilting the dispensing device 110 so as to move the liquid level from the supply cups into the fluid dispensing tubes. In FIGS. 2A and 2B, the supply cups are fed into a common fluid dispensing tube, but those with skill in the art can easily envision designs in which multiple fluid connection tubes are used to provide liquid to the absorbent pad and emanator system. Referring again to FIG. 2A, the dispensing device 110 is depicted in a frontal view. The emanantor 140 is shown, but the absorbent pad is not visible. The wall member 160 is shown and the peelable seal 180 is depicted as a hatch pattern. The vent 190 is depicted above the active supply cups in FIG. 2A.

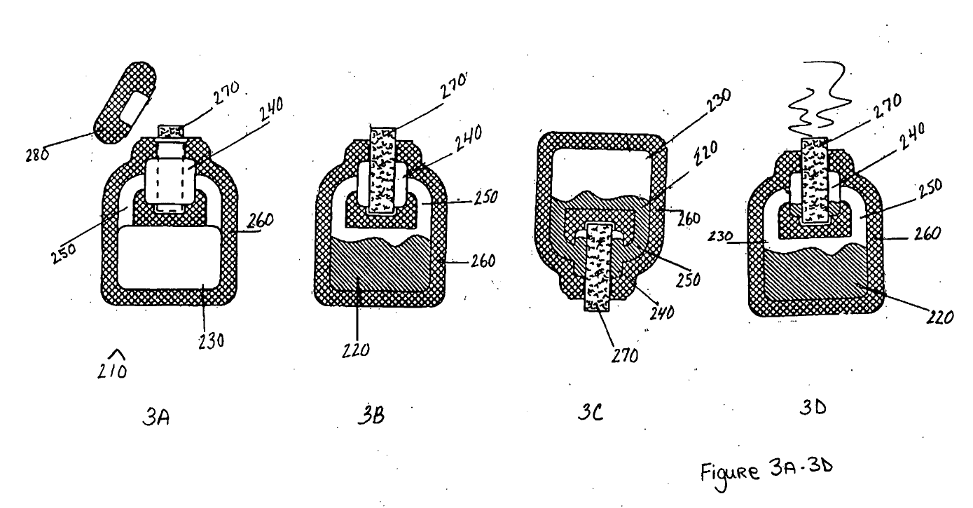

Referring to FIGS. 3A to 3D, a third embodiment of the present invention is presented. In this embodiment the active liquid is provided in a supply cup 230 which is formed by the peripheral union of two wall member 260 wherein the liquid level is below the fluid connecting tubes 250 which provides liquid communication between the supply cups and the absorbent pad and emanator below. The active liquid is dispensed by tilting the dispensing device 210 so as to move the liquid level from the supply cups into the fluid dispensing tubes. In FIGS. 3A-3D, the supply cups are fed into the fluid connecting tubes 250, but those with skill in the art can easily envision designs in which multiple fluid connection tubes are used to provide liquid to the absorbent pad and emanator system. Referring again to FIG. 3A, the dispensing device 210 is depicted in a frontal view. The emanantor 240 is shown and the absorbent pad 270 is in the form of a stick. The union of the two wall members 260 is shown and the removable tab 280 is depicted in the detached position. The tab 280 may be removed by snapping and tearing or any other means known in the art. FIG. 3B illustrates the dispensing device 210 when the tab 280 is removed. FIG. 3C depicts the dispensing device 210 is tilted in the engaged position and the connecting tubes 250 are filled with the active liquid 220 which can contact the emanator 240 thus, commencing the functional operation of said active liquid 220 and the evaporation of the active liquid 220 into the surrounding atmosphere. FIG. 3D depicts the dispensing device 210 in the upright position and the active liquid 220 can still evaporate into the surrounding atmosphere from the absorbent material 270 for a continued effect.

The active liquids of the present invention are desirably fragrance chemicals that can be used alone or in combination with solvents, adjuvants, surfactants, emulsifiers, as well as polymers for encapsulating the fragrance, without departing from the scope of the present invention. The nature and variety of these other ingredients are well-known to those with skill in the art.

Many types of fragrances can be employed in the present invention, the only limitation being the compatibility with the other components being employed. Suitable fragrances include, but are not limited to, fruits such as almond, apple, cherry, grape, pear, pineapple, orange, strawberry, raspberry; musk, flower scents such as lavender-like, rose-like, iris-like, carnation-like. Other pleasant scents include herbal and woodland scents derived from pine, spruce and other forest smells. Fragrances can also be derived from various oils, such as essential oils, or from plant materials such as peppermint, spearmint and the like.

A list of suitable fragrances is provided in U.S. Pat. Nos. 4,145,184; 4,152,272; 4,209,417; 4,534,891 and 5,234,610; the contents of which are incorporated herein by reference in their entirety. Another source of suitable fragrances is found in Perfumes, Cosmetics and Soaps, Second Edition, edited by W. A. Poucher, 1959. Among the fragrances provided in this treatise are acacia, cassie, chypre, cyclamen, fern, gardenia, hawthorn, heliotrope, honeysuckle, hyacinth, jasmine, lilac, lily, magnolia, mimosa, narcissus, freshly-cut hay, orange blossom, orchid, reseda, sweet pea, trefle, tuberose, vanilla, violet, wallflower, and the like.

An olfactory effective amount is understood to mean the amount of compound in perfume compositions the individual component will contribute to its particular olfactory characteristics, but the olfactory effect of the perfume composition will be the sum of the effects of each of the perfumes or fragrance ingredients. Thus the compounds of the invention can be used to alter the aroma characteristics of the perfume composition, or by modifying the olfactory reaction contributed by another ingredient in the composition. The amount will vary depending on many factors including other ingredients, their relative amounts and the effect that is desired.

In another embodiment of the invention, the active ingredient is an insect repelling or insecticide material. These materials are well-known in the art and include, but are not limited to, citronella oil and N,N-diphenyl-m-toluamide, commonly referred to as DEET in the art. Additional information regarding insect repellants and insecticide materials is found in U.S. Pat. Nos. 6,756,046; 6,750,256; 6,749,861; 6,746, 988; 5,439,941; and 5,387,418.

In a particular embodiment, the dispensing device comprises a supply cup 30 which holds an active liquid 20, wherein said supply cup 30 maintains the active liquid 20 supply in isolation from the ambient atmosphere; the supply cup 30 located within the dispensing device 10 replenishes with active liquid 20 of the emanator 40; an emanator 40 which receives active liquid 20 from the supply cup 30 via a tubular element 50, said emanator 40 diffuses said active liquid 20 into the ambient atmosphere by the process of evaporation; and a holding vessel. The holding vessel can be any bottle or container used to store consumer products such as detergent, soap, cleanser, bleach, solvents, paints or the like.

The dispensing device is initiated when a wall of the dispensing device 10 is punctured via the disengagement of a stopper or removal of a barrier, such as a peelable seal, so that the active liquid 20 is free to travel through a tubular element 50 open at each end, to convey the active liquid 20 from the supply cup 30 present to the emanator 40. One end of the tubular element 50 protrudes into the flexible pouch or bag. The tubular element 50 maintains a liquid seal to the pouch or bag via a stopper or barrier at one end of the element. When the stopper is disengaged, the tubular element 50 allows the active liquid 20 to travel and contact the emanator 40. The active liquid 20 can be isolated from the emanator 40 prior to use, by enclosing the liquid in a frangible container within the bag or pouch, or by the use of rupturable or removable barrier strips.

The rate of active liquid 20 dispensing from the supply cup 30 container to emanating surface is primarily controlled by the dimensions of the tubular element 50. A smaller diameter tubular element 50 will result in a slower liquid dispensing rate, when compared to a larger diameter shorter tubular element 50. The active liquid 20 is replenished into the supply cup 30 every time the dispensing device 10 is tilted, resulting in a frequent replenishing of the supply cup 30 and enhanced or prolonged air freshening capability. Typically the tubular element has a diameter of from about 0.1 to about 1.0 centimeters, or more generally from about 0.3 to about 0.7 centimeters and most desirably from about 0.5 centimeters.

The air freshening dispenser operation is initiated by removing or rupturing the barrier between the active liquid 20 present in the supply cup 30 and the emanator 40. The emanator 40 quickly becomes saturated with the active liquid 20 from the supply cup 30. The active liquid 20 is conveyed along the length of the emanator 40. As the active liquid 20 is diffused by the emanator 40 at a slow rate, more liquid is supplied to the emanator 40 at a slow, controlled rate from the supply cup 30. The emanator 40 can in turn be continually replenished with active liquid 20 from the supply cup 30 upon tilting the dispensing device 10.

The tubular element 50 can further comprise a barrier strip which is removed to allow free passage of the active liquid 20 to the emanator 40. The dispensing device 10 is constructed of a flexible material which is capable of collapsing inward in response to the reduced volume, as the active liquid 20 is depleted. The dispensing device 10 material must also be impermeable and compatible with the active liquid. The shape of the dispensing device 10 is generally a planar bag however the shape is not limited to a rectilinear shape. The dispensing device 10 can be constructed by closing or welding the periphery of two polymeric sheets by techniques well-known in the art including heat, adhesives, and ultrasonic energy.

As will be appreciated by the skilled artisan, the barrier strips of this invention are not limited to the examples described. Any means of maintaining the active liquid 20 isolated from the emanator 40 would serve the purpose of the barrier strip. A variety of other methods to keep the active liquid 20 isolated from the emanator 40 prior to use can be used as a barrier for this invention. For example, a complementary rib and groove strip could be welded to the interior walls of the dispensing device 10 bag. The liquid interlocking profile would keep the active liquid 20 isolated from the emanator 40 prior to use. At the time of use, the dispensing device 10 bag walls could be pulled apart, opening the barrier strip, and allowing the active liquid 20 to contact the emanator 40. Any type of rupturable or removable liquid barrier could be used in this invention, to maintain the active liquid 20 isolated from the emanator 40, prior to use. As the active liquid 20 is depleted within the dispensing device 10, the bag collapses inward, in response to the reduced volume, obviating the need for an atmospheric vent. In a particular embodiment, a vent 90 is provided to enable air to enter the reservoir and enable the active liquid to flow more easily. Desirably vents are placed with a flapper or similar mechanism which will substantially reduce spillage from the device when it is tilted or inverted. Each tubular element 50 may have a little hole in the end that when the device is tilted the hole is open to allow air to flow in and out which facilitates the flow of liquid.

The emanator 40 is comprised of any absorbent material 70 capable of conveying the active liquid 20 over a relatively large surface area to aid in its evaporation process. Additionally, the emanator 40 can be fabricated in an aesthetically pleasing or functional shape. When functioning as an air freshener, the emanator 40 of the invention can be formed into a shape characteristic of the scent. For example, the emanator 40 can be shaped as a strawberry, apple, or cherry whereby, the diffused scent would be characteristic of a strawberry, apple, or cherry. A feature of this dispensing device is the compact size of the air freshener, when present on a holding vessel such a consumer goods detergent bottle, the dispensing device does not use any more shelf space than the bottle itself. This allows the area where the bottle is stored to be freshened between uses. With every tilt of the bottle, the supply cup 30 replenishes with active liquid 20 fragrance to the emanator 40. The fragrance will be effective for air freshening, aromatherapy, malodor covering, air sanitizing, insect repellent and the like.

All U.S. patents and patent applications referenced herein are hereby incorporated by reference as if set forth in their entirety. The above description and drawing are provided as specific embodiments of the present invention. Other modifications of this invention will be readily apparent to those skilled in the art and such modifications are understood to be within the scope of this invention.

What is claimed is:

1-7. (canceled)

8. A method for providing an active liquid to the environment comprising tilting a fragrance dispensing device wherein the fragrance dispensing device comprises a replenishable supply cup, wherein said replenishable supply cup maintains an active liquid supply in isolation from the ambient atmosphere; and an emanator which receives active liquid from the replenishable supply cup via a hollow tubular element, said emanator diffuses said active liquid into the ambient atmosphere by the process of evaporation and the supply cup receives the active liquid not absorbed by the emanator flows via the hollow tubular element.

9. (canceled)

10. The method of claim 8, wherein said active liquid is a fragrance.

11. The method of claim 8, wherein said active liquid is an insect repellant.

12. The method of claim 8 further comprising an adhesive backing and a release layer, wherein the release layer is removed to expose said adhesive backing so that the dispenser may be adhered to a surface.
